(19) United States
(12) Patent Application Publication
(10) Pub. No.: US 2007/0016144 A1
(43) Pub. Date: Jan. 18, 2007

(54) Reciprocating syringe assemblies

(76) Inventors: Wilmer L. Sibbitt JR., Albuquerque, NM (US); Adrian A. Michael, Albuquerque, NM (US)

Correspondence Address: Jay A. Stelacone, Esq.;Law Office of Thomas M. Isaacson, 10415 Southern Maryland Blvd., Dunkirk, MD, 20754, US

(21) Appl. No.: 11/446,167

(22) Filed: Jun. 5, 2006

Related U.S. Application Data
(60) Provisional application No. 60/687,580, filed on Jun. 3, 2005.

(51) Int. Cl. A61M 5/315

(52) U.S. Cl. 604/218; 604/191

(57) ABSTRACT

A reciprocating syringe assembly may include a housing having a receptacle structured and arranged to removably receive a syringe and a syringe removably disposed in the receptacle. The syringe comprising a syringe plunger, and the housing may include at least one track. The assembly may include a plunger slidable in the track. The plunger may be mechanically coupled to the syringe plunger such that movement of the plunger in a first direction causes movement of the syringe plunger in a second direction opposite to the first direction and movement of the syringe plunger in the first direction causes movement of the plunger in the second direction.

### CROSS-REFERENCE TO RELATED APPLICATIONS

This application claims the benefit of priority of U.S. provisional application No. 60/687,580, filed on Jun. 3, 2005, the contents of which are incorporated herein by reference in their entirety.

### TECHNICAL FIELD

The present disclosure is directed generally to medical devices and methods. More particularly, the present disclosure is directed to reciprocating syringe assemblies and methods for using such devices in medical procedures.

### BACKGROUND

Syringes are an essential element in the day to day practice of medicine and nursing, but are also essential in industry, laboratory science, research, and animal husbandry. Syringes may be used to inject medications, to aspirate body fluids, to provide vacuum, and/or to transfer fluids. The conventional syringe design most commonly used in medicine consists of a barrel made of plastic and an internal plunger which is moved into or out of the barrel, resulting in pressure or a vacuum, respectively. The difference in pressure between the volume in the syringe and the outside environment are produced by movement of the plunger, resulting in movement of fluid into or out of the syringe. These differences in pressure create the desired effect of a syringe, that is, aspiration or injection.

Injection with a conventional syringe is simple, and uses the powerful flexor muscles of the hand and forearm. Injection with a conventional syringe can usually be easily accomplished with one hand, freeing up the other hand for other necessary tasks or procedures. In an exemplary technique, the second and third fingers (index and middle fingers, respectively) are placed on the finger flange of the syringe and the thumb is placed on the thumb rest of the plunger. The fingers and thumb are brought together resulting in a powerful injection due to contraction of the powerful flexor muscles of the hand and forearm. The ability to use a syringe with one hand in this way and use the other hand for other tasks is important in many complicated procedures.

Aspiration with a conventional syringe usually requires the use of two hands in order to generate the necessary power while maintaining fine control. Generally, this is done by using one hand to control the barrel and the other hand to pull on the thumb rest of the plunger. The two-handed technique uses the muscular strength of both the hands and the arms. Thus, very powerful vacuums with rapid movement of fluid into the syringe can be obtained. This is currently the technique of choice when either fine control of the syringe is required or considerable power is necessary. This is the technique used by most physicians and nurses.

One-handed aspiration with a conventional syringe is possible, but is difficult and awkward. Generally, two techniques can be used. In the first method, the thumb rest of the plunger is grasped by the second and third fingers (index and middle fingers, respectively) and the thumb is placed on the finger flange of the syringe. The fingers are forcefully flexed, while the thumb remains extended. This results in the plunger be pulled out, resulting in an effective aspiration. There are several problems with this method including: 1) fine control of the syringe is effectively lost (which is important when there is a sharp needle in delicate living tissues); 2) the entire syringe tends to rotate, further degrading control; 3) due to the size of the syringe components relative to the dimensions and strength of the human hand, this method is extremely difficult with syringes larger than 10 cc (for example, 20 cc or 60 cc syringes); and 4) the force of aspiration is generated by the weak intrinsic flexors of the hand (without using the powerful flexors of the thumb and forearm), resulting in a weaker aspiration. Thus, this one-handed method is unsatisfactory.

One-handed aspiration can also be accomplished by the alternative thumb method. In this method, the syringe barrel is grasped by the four fingers, and the thumb is placed under the thumb rest of the plunger. With the syringe firmly grasped by the digits, the thumb is extended, resulting in aspiration. Unfortunately, variations of this method are best demonstrated by drug addicts who inject themselves with drugs. The alternative thumb method has several disadvantages: 1) although a degree of control is maintained, it is not the fine control of the fingers, but the more coarse control of the forearm musculature; 2) the power of the aspiration is weak, because it is accomplished by the weak extensors of the thumb; 3) full aspiration is difficult to achieve without changing the handgrip; 4) the syringe is generally pointing toward the operator which is the opposite from the direction typically required in a medical procedure (except for a person injecting themselves with drugs); and 5) when the thumb is extended, the hypothenar tissues are compressed under the syringe, resulting in unpredictable deviation of the needle side of the syringe with some loss of control.

Overall, one-handed aspiration with a conventional syringe is difficult and awkward, resulting in loss of fine control and power during aspiration. With loss of control, there is a higher rate of procedure failure and contamination. With loss of power, speed of aspiration is impaired, especially for viscous fluids. Because of the loss of strength and control with one-handed aspiration, procedures that demand either fine control of the syringe during aspiration or the generation of a powerful vacuum require the use of both hands during aspiration to maintain both strength and control.

U.S. Pat. Nos. 6,245,046 and 6,962,576 as well as U.S. patent application Ser. No. 11/118,258, pending, disclose syringe designs that permit both injection and aspiration with one hand, yet maintain fine motor control and the strength necessary to generate high pressures and vacuums.

It may be desirable to provide reciprocating syringe assemblies configured to convert a non-reciprocating syringe to a reciprocating syringe in order to permit injection and aspiration of fluids or gas using one hand with applications to health care, research, and industry. The present disclosure describes exemplary embodiments of reciprocating syringe assemblies that may solve one or more of the above problems.

### SUMMARY OF THE INVENTION

In accordance with various aspects of the disclosure, a reciprocating syringe assembly may comprise a housing having a receptacle structured and arranged to removably receive a syringe. The housing may include a first track and a second track, wherein a first plunger is slidable in the first track and a second plunger is slidable in the second track. The first plunger may include a thumb rest configured to mate with a thumb rest of a syringe plunger such that motion of the first plunger is imparted to the syringe plunger. The first and second plungers may be mechanically coupled to one another such that movement of the first plunger in a first direction causes movement of the second plunger in a second direction opposite to the first direction and movement of the second plunger in the first direction causes movement of the first plunger in the second direction.

According to some aspects of the disclosure, a reciprocating syringe assembly may comprise a housing having a receptacle structured and arranged to removably receive a syringe and a syringe removably disposed in the receptacle. The syringe comprising a syringe plunger, and the housing may include at least one track. The assembly may include a plunger slidable in the track. The plunger may be mechanically coupled to the syringe plunger such that movement of the plunger in a first direction causes movement of the syringe plunger in a second direction opposite to the first direction and movement of the syringe plunger in the first direction causes movement of the plunger in the second direction.

In accordance with some aspects of the disclosure, a method of reciprocatingly operating a syringe may comprise inserting at least a portion of a syringe in a receptacle of a housing and mechanically coupling a plunger of the syringe with a plunger slidable in a track of the housing such that movement of the plunger in a first direction causes movement of the syringe plunger in a second direction opposite to the first direction and movement of the syringe plunger in the first direction causes movement of the plunger in the second direction. The method may further include urging either the plunger or the syringe plunger in the first direction.

It is to be understood that both the foregoing general description and the following detailed description are exemplary and explanatory only and are not restrictive of the invention.

### BRIEF DESCRIPTION OF THE DRAWINGS

The accompanying drawings, which are incorporated in and constitute a part of this specification, illustrate several exemplary embodiments of the invention and, together with the description, serve to explain the principles of the invention. Unless otherwise noted, the various figures are not necessarily drawn to scale. In the drawings,

FIG. 1A is a bottom view of an exemplary syringe assembly in accordance with various aspects of the disclosure;

FIG. 1B is a top view of the assembly of FIG. 1A;

FIG. 1C is a side view of the assembly of FIG. 1A;

FIG. 1D is a front view of the assembly of FIG. 1A;

FIG. 1E is a back view of the assembly of FIG. 1A;

FIG. 1F is a side view of exemplary driver members for the exemplary syringe assembly of FIG. 1A;

FIG. 1G is a magnified side view of one driver member of FIG. 1F;

FIG. 1H is a bottom view of the exemplary driver members of FIG. 1F;

FIG. 2A is a bottom view of an exemplary syringe assembly in accordance with various aspects of the disclosure;

FIG. 2B is a top view of the exemplary syringe assembly of FIG. 2A;

FIG. 2C is a side view of the exemplary syringe assembly of FIG. 2A;

FIG. 3A is a top view of a conventional syringe;

FIG. 3B is a top view of the exemplary syringe assembly of FIG. 2A holding the syringe of FIG. 3A;

FIG. 3C is a side view of the exemplary syringe assembly of FIG. 2A holding the syringe of FIG. 3A;

FIG. 4A is a bottom view of an exemplary syringe assembly in accordance with various aspects of the disclosure;

FIG. 4B is a top view of the assembly of FIG. 4A;

FIG. 4C is a side view of the assembly of FIG. 4A;

FIG. 4D is a front view of the assembly of FIG. 4A;

FIG. 4E is a back view of the assembly of FIG. 4A;

FIG. 4F is a side view of exemplary driver members for the exemplary syringe assembly of FIG. 4A;

FIG. 4G is a magnified side view of one driver member of FIG. 4F;

FIG. 4H is a bottom view of the exemplary driver members of FIG. 4F;

FIG. 5A is a bottom view of an exemplary assembled syringe assembly in accordance with various aspects of the disclosure;

FIG. 5B is a top view of the exemplary assembled syringe assembly of FIG. 5A;

FIG. 5C is a side view of the exemplary assembled syringe assembly of FIG. 5A;

FIGS. 6A-6D are end views illustrating various plunger driver positions in accordance with various aspects of the disclosure;

FIG. 7A is a bottom view of an exemplary syringe assembly in accordance with various aspects of the disclosure;

FIG. 7B is a top view of the exemplary syringe assembly of FIG. 7A;

FIG. 7C is a side view of the exemplary syringe assembly of FIG. 7A;

FIG. 7D is a front view of the exemplary syringe assembly of FIG. 7A;

FIG. 7E is a back view of the exemplary syringe assembly of FIG. 7A;

FIG. 7F is a side view of exemplary driver members for the exemplary syringe assembly of FIG. 7A;

FIG. 7G is a bottom view of the exemplary driver members of FIG. 7F;

FIG. 8A is a bottom view of an exemplary assembled syringe assembly in accordance with various aspects of the disclosure;

FIG. 8B is a top view of the exemplary assembled syringe assembly of FIG. 8A;

FIG. 8C is a side view of the exemplary assembled syringe assembly of FIG. 8A;

FIG. 9A is a top view of an exemplary syringe;

FIG. 9B is a top view of the exemplary assembled syringe assembly of FIG. 8A holding the syringe of FIG. 9A;

FIG. 9C is a side view of the exemplary assembled syringe assembly of FIG. 8A holding the syringe of FIG. 9A;

FIG. 10A is a bottom view of an exemplary syringe assembly in accordance with various aspects of the disclosure;

FIG. 10B is a top view of the exemplary syringe assembly of FIG. 10A;

FIG. 10C is a side view of the exemplary syringe assembly of FIG. 10A;

FIG. 10D is a front view of the exemplary syringe assembly of FIG. 10A;

FIG. 10E is a back view of the exemplary syringe assembly of FIG. 10A;

FIG. 10F is a side view of exemplary driver members for the exemplary syringe assembly of FIG. 10A;

FIG. 10G is a bottom view of the exemplary driver members of FIG. 10F;

FIG. 11A is a top view of an exemplary syringe;

FIG. 11B is a top view of the exemplary assembled syringe assembly of FIGS. 10A-G holding the syringe of FIG. 11A; and

FIG. 11C is a side view of the exemplary assembled syringe assembly of FIGS. 10A-G holding the syringe of FIG. 11A.

### DETAILED DESCRIPTION

Reference will now be made in detail to embodiments of the invention, examples of which are illustrated in the accompanying drawings. Wherever possible, the same reference numbers will be used throughout the drawings to refer to the same or like parts.

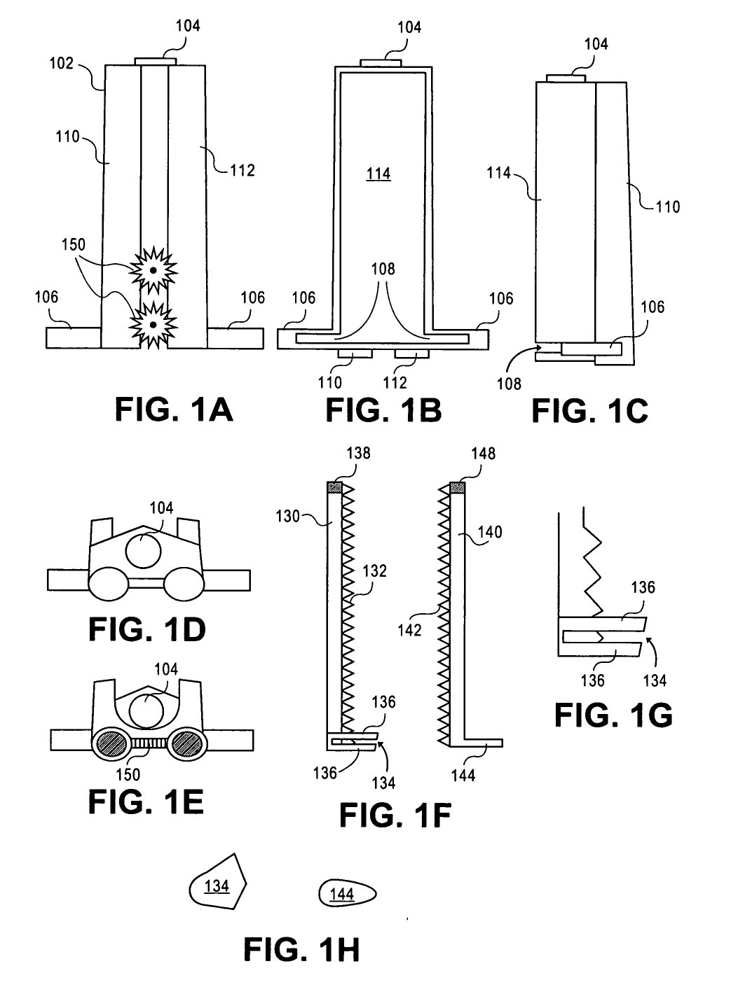

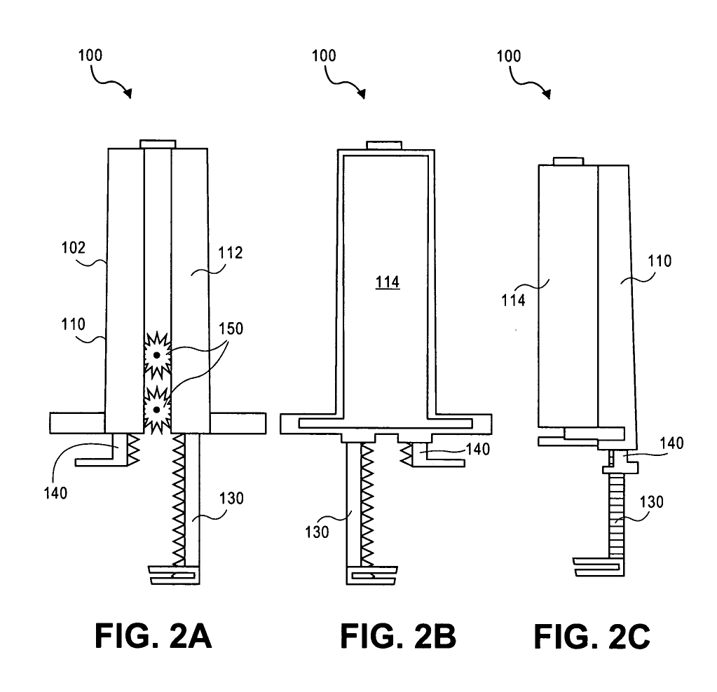

Referring to FIGS. 1A-1H and 2A-2C, an exemplary reciprocating syringe assembly 100 in accordance with various aspects of the disclosure may include a syringe assembly housing 102 with a receptacle 114 configured to receive a syringe and two tracks, or housings, 110, 112 configured to receive a pair of drivers 130, 140 of the reciprocating syringe assembly 100. The first track 110 receives a plunger driver 123 (engaging syringe plunger driver) configured to engage a syringe plunger. The second track 112 receives a secondary, non-engaging plunger driver 140. According to various aspects, the first and second tracks 110, 112 may be substantially parallel to one another as shown in FIG. 1A. In some aspects, the first and second tracks 110, 112 may be non-parallel. As illustrated in FIG. 1B, the first and second tracks 110, 112 may be inferior to the syringe assembly housing 102 in order to decrease the profile of the syringe assembly 100 with syringe between the index and middle fingers.

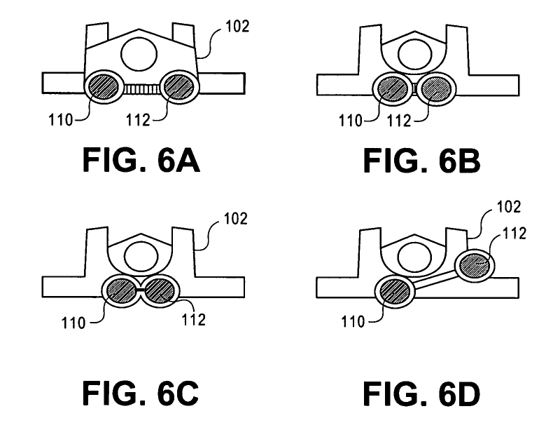

According to some embodiments, the tracks 110, 112 may be placed in various positions, including positions that are not inferior to the syringe assembly housing 102, such as, for example, the various positions illustrated in FIGS. 6A-6D. For example, FIG. 6A shows the tracks 110, 112 reasonably far apart; FIG. 6B shows the tracks 110, 112 substantially as close as possible; FIG. 6C shows the tracks 110, 112 substantially contiguous; and FIG. 6D shows the first track 110 inferior to the housing 102 and the second track 112 extending outside the housing 102. It should be appreciated that in some aspects, diagonal tracks and other variations may be appropriate. In FIGS. 6A-6D, the tracks 110, 112 are roughly cylindrical, but in some aspects, the tracks 110, 112 could be any shape, configuration, and/or cross-sectional structure including rectangular, oval, or other geometric structure that accommodates movement along a substantially rigid longitudinal track. The tracks 110, 112 need not be parallel to function, but can be also be non-parallel.

The reciprocating syringe assembly 100 may comprise a needle fitting holder 104 configured to mate with or accommodate an exterior surface of a syringe needle fitting. The needle fitting holder 104 does not require a Luer or other fitting. The needle fitting holder 104 may function as an exterior holder to control an inserted syringe. Although shown in FIGS. 1D and 1E as a complete circular opening in the syringe assembly 100, it should be appreciated that, in various aspects, the needle fitting holder 104 may be an incomplete circle or noncircular.

The reciprocating syringe assembly 100 may include a pair of finger flanges 106 to facilitate control of a syringe. The finger flanges 106 may accommodate the index and middle fingers, which may remain in one position throughout aspiration and injection by a syringe. The reciprocating syringe assembly 100 may comprise a holder portion 108 structured and arranged to receive finger flanges of a syringe. Although FIG. 1B shows the holder portion 108 integrated with the finger flanges 106, it should be appreciated that the holder portion 108 does not have to be integrated with the finger flanges 106, but could comprise slits or another mating structure on the syringe assembly housing configured to accommodate the finger flanges of the syringe and fixedly hold the syringe barrel.

The reciprocating syringe assembly 100 may include a plunger driver complex comprising two drivers 130, 140. The first driver 130 may be an engaging syringe plunger driver configured to drive the syringe plunger, for example, with teeth 132 configured to engage one or more driver gears. The first driver 130 may further include a thumb rest 134 configured to engage and/or bind a thumb rest of a syringe plunger, thereby permitting force to be transferred from the first driver 130 to the plunger of the syringe. According to various aspects, the thumb rest 134 may comprise two substantially parallel flanges 136 structured and arranged to sandwich a thumb rest of a syringe plunger. One skilled in the art would understand that other known structures configured to engage and/or bind a syringe plunger can be readily substituted for the parallel flanges.

The second driver 140 may be a secondary plunger driver mechanically coupled with the first driver 130, for example, via one or more driver gears 150. The second driver 140 may include teeth 142 configured to engage the one or more gears 150. The second driver 140 may also comprise a thumb rest 144 configured to be urged by a user's thumb. As shown in FIG. 1H, the thumb rests 134, 144 may be asymmetrical. In some aspects, the thumb rests may be symmetrical. In some aspects of the invention, the first driver 130 and/or the second driver 140 may include a friction member 138, 148 configured to create a drag force to thereby help stabilize the assembly 100.

The first and second drivers 130, 140 may oppose the one or more gears 150 and share the one or more gears 150 kinetically. The one or more gears 150 may be coupled to the syringe assembly 100 and configured to engage the teeth 132, 142 of the first and second plunger drivers 130, 140. The one or more gears 150 may be in direct contact with both plunger drivers 130, 140. Thus, each of the driver tracks 110, 112 may include a window, a slot, or another type of access (not shown) configured to permit the one or more gears 150 to interdigitate (or mechanically interact) with the teeth 132, 142 on the plunger drivers 130, 140.

The components of the reciprocating syringe assembly 100 illustrated in FIGS. 1A-1H are shown assembled in FIGS. 2A-2C. In operation, when one of first and second drivers 130, 140 is depressed by a user's thumb, the other one of the first and second drivers 130, 140 is driven in the opposite direction in a reversible fashion. Thus, reciprocating syringe assembly 100 comprises a pair of reciprocating plunger drivers 130, 140 that are thumb operated.

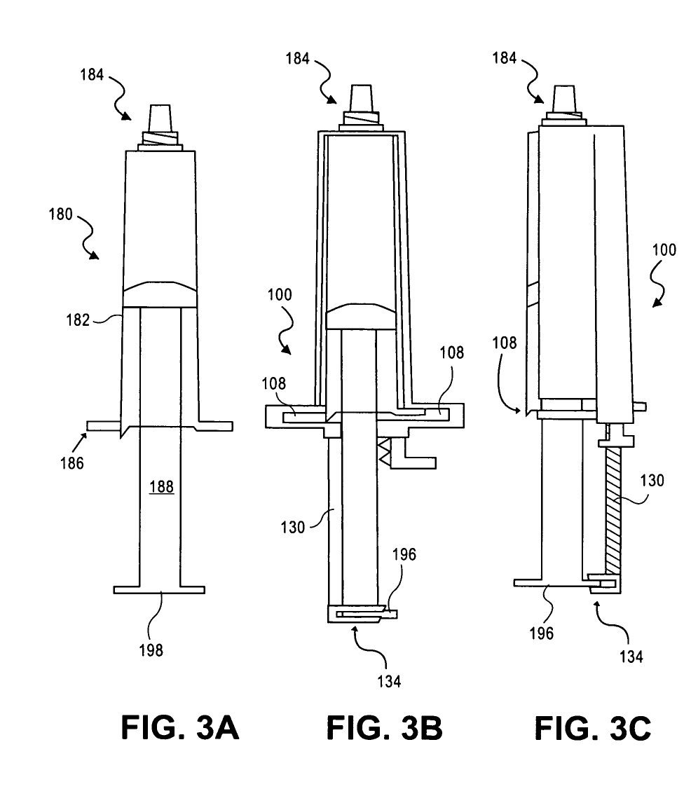

Referring now to FIGS. 3A-3C, a syringe 180 may be inserted into the receptacle 114 of the reciprocating syringe assembly 100. The syringe 180 may comprise any conventional or specialty syringe. The syringe 180 may include a barrel portion 182, a needle fitting 184, a finger flange 186, and a plunger 188 as is well known in the art. The plunger 188 may include a thumb rest 198. The syringe needle fitting 184 may protrude from the receptacle 114 of the reciprocating syringe assembly 100 and be held substantially firmly in place by the needle fitting holder 104.

As shown in FIG. 3, the finger flange 192 of the syringe 190 may be held in place by the holder portion 108 of the reciprocating syringe assembly 100, and the thumb rest 134 of the first driver 130 may mate with the thumb rest 196 of the syringe 190. In operation, the thumb rest 144 of the second driver 140 may be pressed to aspirate, and the thumb rest 134 of the first driver 130 may be pressed to inject.

As a result, the reciprocating syringe assembly 100 may reversibly convert a conventional or specialty non-reciprocating syringe 180 into a reciprocating syringe. In the assembly 100, the non-reciprocating syringe 180 can be used in a reciprocating fashion and then removed from the syringe assembly 100 for processing. It should be appreciated that the syringe assembly 100 can be made in one piece, for example, by injection molding, and then adding the one or more gears 150, or it can be made in pieces and subsequently assembled together.

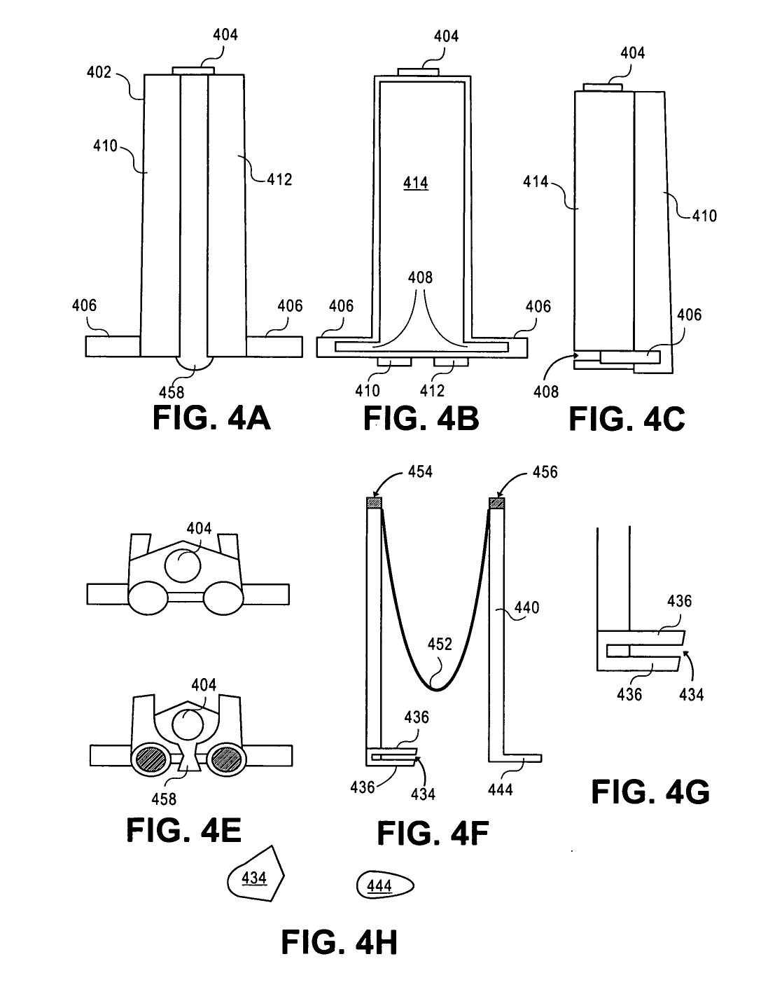

Referring now to FIGS. 4A-4H, an exemplary reciprocating syringe assembly 400 in accordance with various aspects of the disclosure may include a syringe assembly housing 402 with a receptacle 414 configured to receive a syringe and two tracks, or housings, 410, 412. The two tracks 410, 412 are configured to receive a pair of drivers 430, 440 of the reciprocating syringe assembly 100. The first track 410 receives a plunger driver 430 configured to engage a syringe plunger. The second track 412 receives a secondary, non-engaging plunger driver 440. According to various aspects, the first and second tracks 410, 412 may be substantially parallel to one another as shown in FIG. 4A. In some aspects, the first and second tracks 410, 412 may be non-parallel. As illustrated in FIG. 4B, the first and second tracks 410, 412 may be inferior to the syringe assembly housing 402 in order to decrease the profile of the syringe assembly 400 with syringe between the index and middle fingers.

According to some embodiments, the tracks 410, 412 may be placed in various positions, including positions that are not inferior to the syringe assembly housing 402, such as, for example, the various positions illustrated in FIGS. 6A-6D. In FIGS. 6A-6D, the tracks 110, 112 are roughly cylindrical, but in some aspects, the tracks 410, 412 could be any number of shapes, configurations, and cross-sectional structures including rectangular, oval, or other geometric structure that accommodates movement along a substantially rigid longitudinal track. The tracks 410, 412 need not be parallel to function, but can be also be non-parallel.

The reciprocating syringe assembly 400 may comprise a needle fitting holder 404 configured to mate with or accommodate an exterior surface of a syringe needle fitting. The needle fitting holder 404 does not require a Luer or other fitting. The needle fitting holder 404 may function as an exterior holder to control an inserted syringe. Although shown in FIGS. 4D and 4E as a complete circular opening in the syringe assembly 400, it should be appreciated that, in various aspects, the needle fitting holder 404 may be an incomplete circle or noncircular.

The reciprocating syringe assembly 400 may include a pair of finger flanges 406 to facilitate control of a syringe. The finger flanges 406 may accommodate the index and middle fingers, which may remain in one position throughout aspiration and injection by a syringe. The reciprocating syringe assembly 400 may comprise a holder portion 408 structured and arranged to receive finger flanges of a syringe. Although FIG. 4B shows the holder portion 408 integrated with the finger flanges 406, it should be appreciated that the holder portion 408 does not have to be integrated with the finger flanges 406, but could comprise slits or another mating structure on the syringe assembly housing configured to accommodate the finger flanges of the syringe and fixedly hold the syringe barrel.

The reciprocating syringe assembly 400 may include a plunger driver complex comprising two drivers 430, 440. The first driver 430 may be an engaging syringe plunger driver configured to drive the syringe plunger, for example, via a pulley assembly. The first driver 430 may further include a thumb rest 434 configured to engage and/or bind a thumb rest of a syringe plunger, thereby permitting force to be transferred from the first driver 430 to the plunger of the syringe. According to various aspects, the thumb rest 434 may comprise two substantially parallel flanges 436 structured and arranged to sandwich a thumb rest of a syringe plunger. One skilled in the art would understand that other known structures configured to engage and/or bind a syringe plunger can be readily substituted for the parallel flanges.

The second driver 440 may be a secondary plunger driver mechanically coupled with the first driver 430, for example, via a pulley assembly 450. The pulley assembly 450 may comprise a driveline 452 having one end connected near a distal end 454 of the first driver 430 and another end connected near a distal end 456 of the second driver 440. The pulley assembly 450 may be a self-threading pulley. For example, the driveline 452 may extend over a pulley surface 458 associated with the housing 402 and configured to guide motion of the driveline 452 during reciprocation of the drivers 430, 440. The pulley surface 458 may have any configuration known to persons skilled in the art, including static and/or dynamic elements. The driveline 452 may be monofilament, but may comprise of any substance with adequate strength and flexibility.

In accordance with various aspects, the first and second drivers 430, 440 and the driveline 452 may be assembled from conventional or specially molded parts. In such exemplary aspects, the driveline may comprise a continuous columnar filament or other continuous material, and the attachment of the driveline 452 to the drivers 430, 440 can be achieved in a number of ways. For example, the attachment can be achieved: 1) by welding or cementing the driveline to the drivers, 2) by creating a hole in the drivers through which the driveline may be placed and then fixed by tying, welding, cementing, or distorting the driveline with heat or mechanical means so that it does not pull through the hole, 3) by the means of mechanical clamps that clamp to the drivers and clamp to the driveline causing attachment, 4) by hooks, rings, grommets, compressive mechanisms, or other similar devices on the drivers to which the driveline may be attached by tying welding, crimping, or friction, or 5) by creating male and female connectors (or other complementary connectors) that may attach to either the driveline or the drivers and then are connected to each other by pushing the male and female connectors together. These driveline/driver connectors and other connectors could be attached to and made in the drivers and/or driveline after each piece is produced, or could be molded as an integrated attachment to each component when that component is molded or extruded or otherwise produced.

According to various aspects, the first and second drivers 430, 440 and the driveline 452 may be molded as one integrated part, for example, with the choice of an appropriate plastic. In some aspects, an appropriate temperature resistant driveline could be placed into the mould, and then the two the drivers molded around it, resulting in a high temperature bond between the drivers and driveline and an integrated device. The drivers themselves could be solid, strutted (as in conventional syringes), columnar, hollow, or any other geometrical shape, as long as they accommodate and are complementary to the design of a syringe barrel and/or the first and second tracks 410, 412.

The second driver 440 may comprise a thumb rest 444 configured to be urged by a user's thumb. As shown in FIG. 4H, the thumb rests 434, 444 may be asymmetrical. In some aspects, the thumb rests may be symmetrical. In some aspects of the invention, the first driver 430 and/or the second driver 440 may include a friction member 438, 448 configured to create a drag force to thereby help stabilize the assembly 400.

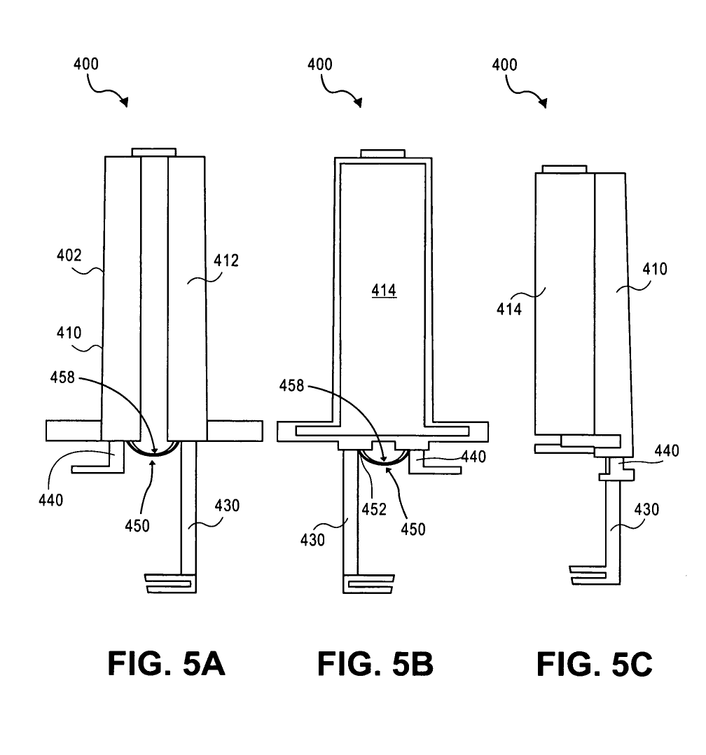

The components of the reciprocating syringe assembly 400 illustrated in FIGS. 4A-4H are shown assembled in FIGS. 5A-5C. In operation, when one of first and second drivers 430, 440 is depressed by a user's thumb, the other one of the first and second drivers 430, 440 is driven in the opposite direction in a reversible fashion. Thus, reciprocating syringe assembly 400 comprises a pair of reciprocating plunger drivers 430, 440 that are thumb operated.

The insertion of a syringe into the assembly 400 and operation of the reciprocating syringe assembly 400 with syringe is similar to that described above in connection with FIGS. 3A-3C. Thus, the reciprocating syringe assembly 400 reversibly converts a non-reciprocating syringe into a fully functional pulley-driven reciprocating syringe, which can be used in a reciprocating fashion and then removed from the syringe assembly for processing.

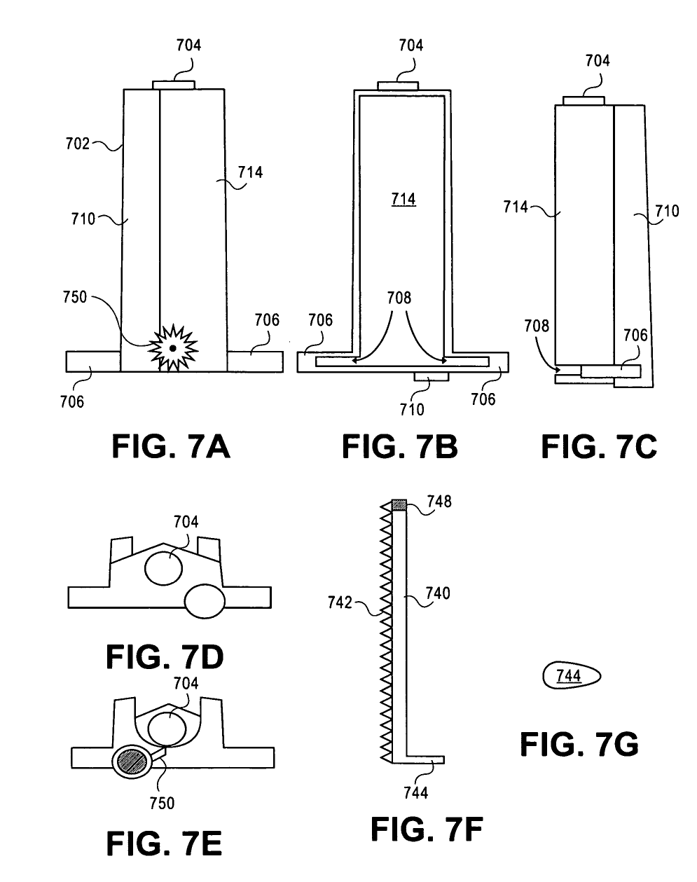

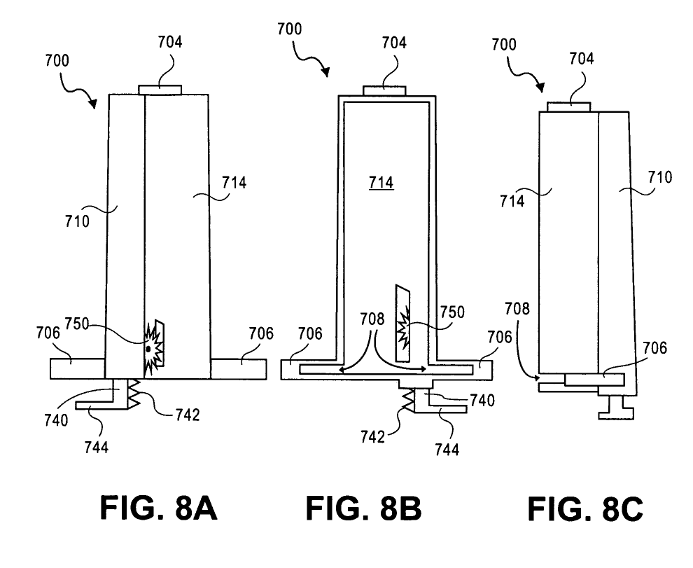

According to some aspects of the disclosure, a reciprocating syringe assembly does not need to have two drivers. For example, an exemplary reciprocating syringe assembly 700 may use the plunger of a syringe itself as one driver. Referring to FIGS. 7A-7G and 8A-8C, the exemplary reciprocating syringe assembly 700 in accordance with various aspects of the disclosure may include a syringe assembly housing 702 with a receptacle 714 configured to receive a syringe and a track, or housing, 710 configured to receive a driver 740 of the reciprocating syringe assembly 700.

The reciprocating syringe assembly 700 may comprise a needle fitting holder 704 configured to mate with or accommodate an exterior surface of a syringe needle fitting. The needle fitting holder 704 does not require a Luer or other fitting. The needle fitting holder 704 may function as an exterior holder to control an inserted syringe. Although shown in FIGS. 7D and 7E as a complete circular opening in the syringe assembly 700, it should be appreciated that, in various aspects, the needle fitting holder 704 may be an incomplete circle or noncircular.

The reciprocating syringe assembly 700 may include a pair of finger flanges 706 to facilitate control of a syringe. The finger flanges 706 may accommodate the index and middle fingers, which may remain in one position throughout aspiration and injection by a syringe. The reciprocating syringe assembly 700 may comprise a holder portion 708 structured and arranged to receive finger flanges of a syringe. Although FIG. 7B shows the holder portion 708 integrated with the finger flanges 706, it should be appreciated that the holder portion 708 does not have to be integrated with the finger flanges 706, but could comprise slits or another mating structure on the syringe assembly housing configured to accommodate the finger flanges of the syringe and fixedly hold the syringe barrel.

The reciprocating syringe assembly 700 may include a driver 740 having teeth 742 configured to engage one or more gears 750. The driver 740 may also comprise a thumb rest 744 configured to be urged by a user's thumb. As shown in FIG. 7H, the thumb rest 744 may be asymmetrical. In some aspects, the thumb rests may be symmetrical. In some aspects of the invention, the driver 740 may include a friction member 748 configured to create a drag force to thereby help stabilize the assembly 700.

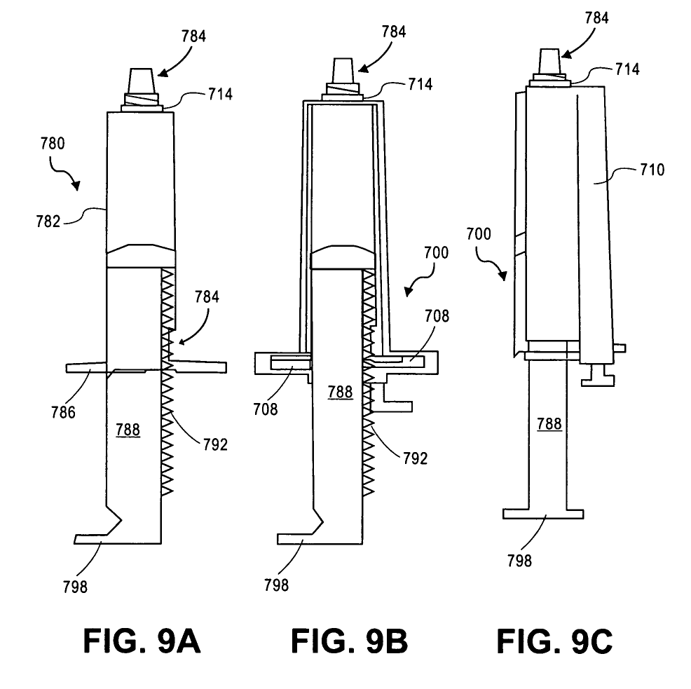

The components of the reciprocating syringe assembly 100 illustrated in FIGS. 7A-7G are shown assembled in FIGS. 8A-8C. Referring now to FIGS. 9A-9C, a syringe 780 may be inserted into the receptacle 714 of the reciprocating syringe assembly 700. The syringe 780 may comprise any conventional or specialty syringe. The syringe 780 may include a barrel portion 782, a needle fitting 784, a finger flange 786, and a plunger 788 as is well known in the art. The plunger 788 may include a thumb rest 798. The syringe needle fitting 784 may protrude from the receptacle 714 of the reciprocating syringe assembly 700 and be held substantially firmly in place by the needle fitting holder 704. As shown in FIGS. 9B and 9C, the finger flange 792 of the syringe 790 may be held in place by the holder portion 708 of the reciprocating syringe assembly 700.

The syringe plunger 788 may include teeth 792 configured to engage the one or more gears 750 and a window 794 structured and arranged to permit the one or more gears 750 to interdigitate with the teeth 792. Thus, the driver 740 and the syringe plunger 788 may oppose the one or more gears 750 and share the one or more gears 750 kinetically. The one or more gears 750 may be coupled to the syringe assembly 700 and configured to engage the teeth 742 of the driver 740 and the teeth 792 of the syringe plunger 788. The one or more gears 150 may be in direct contact with the driver 740 and the syringe plunger 788.

In operation, when the driver 740 is depressed by a user's thumb, the syringe plunger 788 is driven in the opposite direction in a reversible fashion. Likewise, when the syringe plunger 788 is depressed by a user's thumb, the driver 740 is driven in the opposite direction in a reversible fashion. Thus, reciprocating syringe assembly 700 comprises a pair of reciprocating plunger drivers 740, 788 that are thumb operated. In operation, the thumb rest 744 of the driver 740 may be pressed to aspirate, and the thumb rest 798 of the syringe plunger 788 may be pressed to inject.

As a result, the reciprocating syringe assembly 700 may reversibly convert a conventional or specialty non-reciprocating syringe 780 into a reciprocating syringe. In the assembly 700, the non-reciprocating syringe 780 can be used in a reciprocating fashion and then removed from the syringe assembly 700 for processing. It should be appreciated that the syringe assembly 700 can be made in one piece, for example, by injection molding, and then adding the one or more gears 750, or it can be made in pieces and subsequently assembled together.

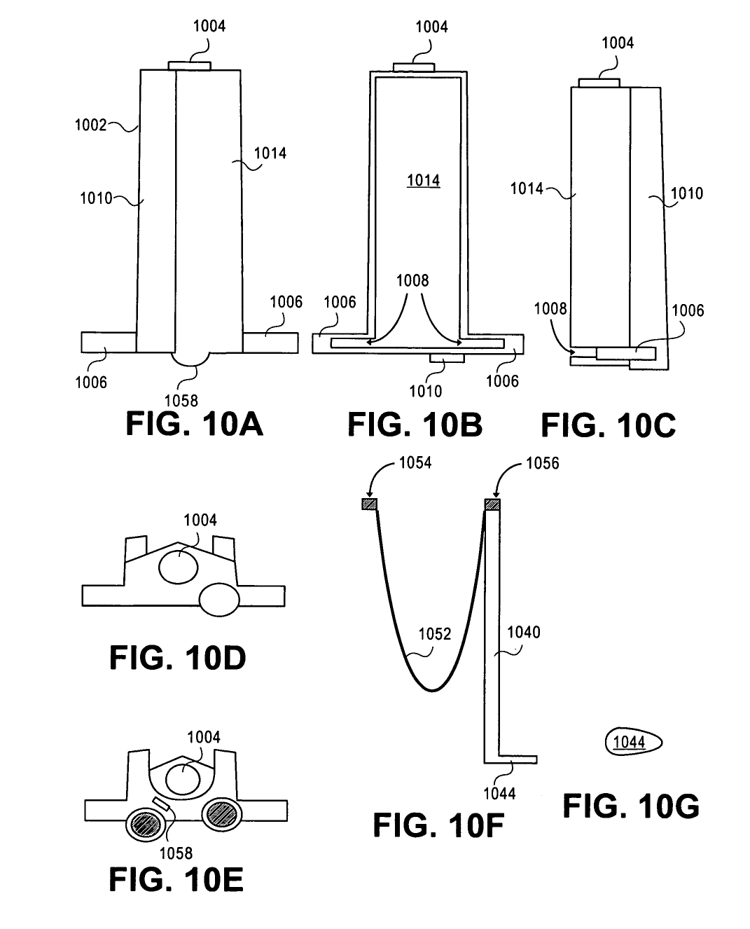

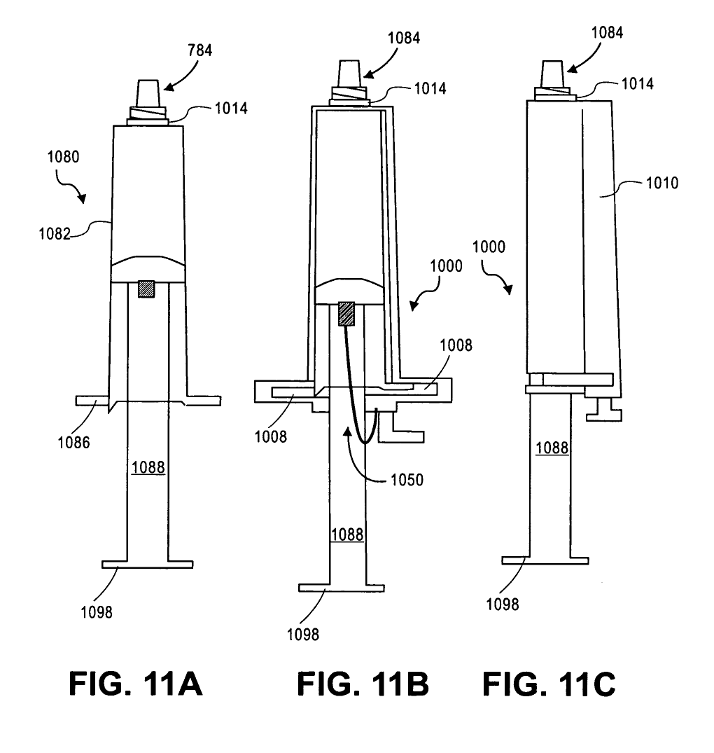

In another exemplary embodiment of a reciprocating syringe assembly that does not need to have two drivers, as illustrated in FIGS. 10A-10G and 11A-11C, the exemplary reciprocating syringe assembly 1000 may include a syringe assembly housing 1002 with a receptacle 1014 configured to receive a syringe and a track, or housing, 1010 configured to receive a driver 1040 of the reciprocating syringe assembly 1000.

The reciprocating syringe assembly 1000 may comprise a needle fitting assembly 1004 configured to mate with or accommodate an exterior surface of a syringe needle fitting. The needle fitting assembly 1004 does not require a Luer or other fitting. The needle fitting assembly 1004 may function as an exterior holder to control an inserted syringe. Although shown in FIGS. 10D and 10E as a complete circular opening in the syringe assembly 1000, it should be appreciated that, in various aspects, the needle fitting assembly 1004 may be an incomplete circle or noncircular.

The reciprocating syringe assembly 1000 may include a pair of finger flanges 1006 to facilitate control of a syringe. The finger flanges 1006 may accommodate the index and middle fingers, which may remain in one position throughout aspiration and injection by a syringe. The reciprocating syringe assembly 1000 may comprise a holder portion 1008 structured and arranged to receive finger flanges of a syringe. Although FIG. 10B shows the holder portion 1008 integrated with the finger flanges 1006, it should be appreciated that the holder portion 1008 does not have to be integrated with the finger flanges 1006, but could comprise slits or another mating structure on the syringe assembly housing configured to accommodate the finger flanges of the syringe and fixedly hold the syringe barrel.

The reciprocating syringe assembly 1000 may include a driver 1040 having teeth 1042 configured to engage the one or more gears 1050. The driver 1040 may also comprise a thumb rest 1044 configured to be urged by a user's thumb. As shown in FIG. 10H, the thumb rest 1044 may be asymmetrical. In some aspects, the thumb rests may be symmetrical. In some aspects of the invention, the driver 1040 may include a friction member 1048 configured to create a drag force to thereby help stabilize the assembly 1000.

The components of the reciprocating syringe assembly 1000 illustrated in FIGS. 10A-10G are shown assembled in FIGS. 11A-11C with a syringe 1080 inserted into the receptacle 1014 of the reciprocating syringe assembly 1000. The syringe 1080 may comprise any conventional or specialty syringe. The syringe 1080 may include a barrel portion 1082, a needle fitting 1084, a finger flange 1086, and a plunger 1088 as is well known in the art. The plunger 1088 may include a thumb rest 1098. The syringe needle fitting 1084 may protrude from the receptacle 1014 of the reciprocating syringe assembly 1000 and be held substantially firmly in place by the needle fitting assembly 1004. As shown in FIGS. 11B and 11C, the finger flange 1092 of the syringe 1090 may be held in place by the holder portion 1008 of the reciprocating syringe assembly 1000.

The driver 1040 may be mechanically coupled with the syringe plunger 1088, for example, via a pulley assembly 1050. The pulley assembly 1050 may comprise a driveline 1052 having one end connected near a distal end 1054 of the syringe plunger 1088 and another end connected near a distal end 1056 of the driver 1040. According to some aspects, the plunger 1088 may comprise a fitting 1096 configured to mate with a fitting 1046 at the one end of the driveline 1052. The driveline 1052 may be coupled to the driver 1040 in a similar manner. It should be appreciated that the aforementioned couplings may be replaced with any appropriate coupling known in the art.

The pulley assembly 1050 may be a self-threading pulley. For example, the driveline 1052 may extend over a pulley surface 1058 configured to guide motion of the driveline 1052 during reciprocation of the plunger 1088 and driver 1040. The pulley surface 1058 may have any configuration known to persons skilled in the art, including static and/or dynamic elements. The driveline 1052 may be monofilament, but may comprise of any substance with adequate strength and flexibility.

In operation, when the driver 1040 is depressed by a user's thumb, the syringe plunger 1088 is driven in the opposite direction in a reversible fashion. Likewise, when the syringe plunger 1088 is depressed by a user's thumb, the driver 1040 is driven in the opposite direction in a reversible fashion. Thus, reciprocating syringe assembly 1000 comprises a pair of reciprocating plunger drivers 1040, 1088 that are thumb operated. In operation, the thumb rest 1044 of the driver 1040 may be pressed to aspirate, and the thumb rest 1098 of the syringe plunger 1088 may be pressed to inject.

As a result, the reciprocating syringe assembly 1000 may reversibly convert a conventional or specialty non-reciprocating syringe 1080 into a reciprocating syringe. In the assembly 1000, the non-reciprocating syringe 1080 can be used in a reciprocating fashion and then removed from the syringe assembly 1000 for processing. It should be appreciated that the syringe assembly 1000 can be made in one piece, for example, by injection molding, and then adding the one or more gears 1050, or it can be made in pieces and subsequently assembled together.

It should be appreciated that plunger locks may be used to prevent unintended injection or loss of fluid contained within a syringe, or alternatively to maintain pressure or vacuum in a syringe. Any conventional plunger locks will function on the reciprocating syringe plunger driver in the same fashion as a conventional syringe. However, the fact that the reciprocating syringe assembly is a double plunger driver where the drivers are mechanically linked in a reciprocating fashion provides some unique aspects of the reciprocating syringe assembly that are different from conventional syringes. In a conventional syringe, a plunger lock may include: 1) a rigid spacer device that fits between the thumb rest of the plunger and the syringe barrel (usually fixes the plunger so that it cannot be depressed), 2) a holder that mechanically binds both the syringe barrel and plunger in a fixed position (prevents both depression and extraction of the plunger), and/or 3) a locking plunger, were there is a fitting on the plunger or barrel that when the plunger is rotated or the fitting is actuated the plunger binds and locks to the barrel. In a conventional syringe, these locking devices function on the dominant plunger, as there is no accessory plunger.

However, in the reciprocating syringe assembly, since the engaging plunger driver is mechanically bound to the non-engaging plunger driver, control of the non-engaging plunger driver will control the engaging plunger driver, and thus, the plunger of the syringe. Thus, the reciprocating syringe assembly is unique in that the locking mechanisms can be applied specifically to the non-engaging plunger driver instead of the plunger or the engaging plunger driver. Thus, conventional locking plungers and plunger locks can be applied to the non-engaging plunger driver to control the engaging plunger driver.

One use of the reciprocating syringe assemblies 100, 400, 700, 1000 may be to generate vacuum for various procedures. When generating vacuum with the reciprocating syringe assembly, the non-engaging plunger driver may be depressed with the thumb, causing the engaging plunger driver to move proximally, thus resulting in aspiration by the plunger of the syringe. As the non-engaging plunger driver is depressed during aspiration, the thumb rest of the non-engaging plunger driver becomes closer to the syringe assembly body, including the finger flanges, providing a unique opportunity for creating a locking mechanism. In this construct, a locking mechanism may be placed on the non-engaging plunger driver close to the thumb rest so that as the thumb rest approaches the syringe assembly body, the locking mechanism on the plunger driver interdigitates with the corresponding mechanism on the syringe assembly body, resulting in locking of the non-engaging plunger driver to the syringe assembly body. Since the engaging plunger driver and syringe plunger are both mechanically linked to the non-engaging plunger driver in a reciprocating fashion, fixing the non-engaging plunger driver to the syringe assembly body fixes the engaging plunger driver and syringe plunger in the aspiration mode, creating a constant vacuum. This may be useful for fine needle aspiration biopsy as well as other uses.

Mechanisms for accomplishing this locking of the accessory plunger to the barrel complex can be accomplished: 1) by the means of mechanical clamps which clamp to the plunger and clamp to syringe assembly body, 2) by hooks, rings, grommets, compressive mechanisms, or other similar devices, 3) by creating male and female connectors (or other complementary connectors) that are connected to each other by pushing the male and female connectors together and reversibly releasing them, and/or 4) by locking mechanisms on the syringe assembly body and plunger drivers that actuate by rotating the plunger driver.

In accordance with various aspects of the disclosure, the syringe assemblies 100, 400, 700, 1000 may reversibly convert a non-reciprocating syringe to a reciprocating syringe, and thus, permit injection and aspiration of fluids or gas using one hand with applications to health care, research, and industry. These holders 100, 400, 700, 1000 may permit the index and middle fingers to stay in one position during aspiration and injection, while the thumb only need move laterally to the alternative plunger in order to change the direction of aspiration or injection. The resulting syringe assembly may be highly stable since only the thumb position changes, and very powerful vacuums or pressures can be developed since the powerful flexors of the fingers, thumb, and forearm may be used for both aspiration and injection.

According to various aspects, the syringe assemblies 100, 400, 700, 1000 are designed to be used with one hand, but can be used in all cases where standard syringes are used. These syringe assemblies may be effective in medical procedures when one-handed injection/aspiration is required (such as cardiac catheterization, emergency procedures, certain types of surgery, pediatric and veterinary procedures and in those handicapped individuals who can only use one hand).

It should be appreciated that this disclosure includes reciprocating syringe assemblies with parallel or non-parallel barrel or tracks, one or two drivers, stopper/drag devices, asymmetrical or symmetrical thumb rests, gear-driven mechanisms, pulley driven mechanisms, pneumatics/hydraulic mechanisms, spring-and-ratchet mechanisms, pulley-driveline plunger complexes, self-threading and enclosed pulleys, methods to construct the barrel complex, external frames or housings to create the barrel complex, and locking plungers.

It will be apparent to those skilled in the art that various modifications and variations can be made to the syringe assemblies and methods of the present disclosure without departing from the scope of the invention. Other embodiments of the invention will be apparent to those skilled in the art from consideration of the specification and practice of the invention disclosed herein. It is intended that the specification and examples be considered as exemplary only.

What is claimed is:

1. A reciprocating syringe assembly, comprising:
a housing having a receptacle structured and arranged to removably receive a syringe, said housing including a first track and a second track;
a first plunger slidable in said first track, said first plunger including a thumb rest configured to mate with a thumb rest of a syringe plunger such that motion of said first plunger is imparted to said syringe plunger; and
a second plunger slidable in said second track, said first and second plungers being mechanically coupled to one another such that movement of the first plunger in a first direction causes movement of the second plunger in a second direction opposite to the first direction and movement of the second plunger in the first direction causes movement of the first plunger in the second direction.

2. The assembly of claim 1, further comprising a syringe removably inserted in said receptacle

3. The assembly of claim 2, further comprising an opening in said receptacle configured to receive a needle fitting of said syringe therethrough.

4. The assembly of claim 1, further comprising a pulley assembly mechanically coupling said first and second plungers.

5. The assembly of claim 1, further comprising at least one gear mechanically coupling said first and second plungers.

6. The assembly of claim 1, wherein the said thumb rest of said first plunger comprises a pair of substantially parallel flanges configured to sandwich said thumb rest of said syringe plunger.

7. A reciprocating syringe assembly, comprising:
a housing having a receptacle structured and arranged to removably receive a syringe, said housing including at least one track;
a syringe removably disposed in said receptacle, said syringe comprising a syringe plunger; and
a plunger slidable in said track, said plunger being mechanically coupled to said syringe plunger such that movement of the plunger in a first direction causes movement of the syringe plunger in a second direction opposite to the first direction and movement of the syringe plunger in the first direction causes movement of the plunger in the second direction.

8. The assembly of claim 7, further comprising an opening in said receptacle configured to receive a needle fitting of said syringe therethrough.

9. The assembly of claim 7, further comprising a pulley assembly mechanically coupling said plunger and said syringe plunger.

10. The assembly of claim 7, further comprising at least one gear mechanically coupling said plunger and said syringe plunger.

11. The assembly of claim 7, wherein said at least one track comprises a first track and a second track, said plunger slidable in said first track, the assembly further comprising:
a second plunger slidable in said second track, said first and second plungers being mechanically coupled to one another such that movement of the first plunger in a first direction causes movement of the second plunger in a second direction opposite to the first direction and movement of the second plunger in the first direction causes movement of the first plunger in the second direction.

12. The assembly of claim 11, wherein said second plunger includes a thumb rest configured to mate with a thumb rest of the syringe plunger such that motion of said second plunger is imparted to said syringe plunger.

13. The assembly of claim 12, wherein the said thumb rest of said second plunger comprises a pair of substantially parallel flanges configured to sandwich said thumb rest of said syringe plunger.

14. The assembly of claim 11, further comprising an opening in said receptacle configured to receive a needle fitting of said syringe therethrough.

15. The assembly of claim 11, further comprising a pulley assembly mechanically coupling said plunger and said second plunger.

16. The assembly of claim 11, further comprising at least one gear mechanically coupling said plunger and said second plunger.

17. A method of reciprocatingly operating a syringe, comprising:
inserting at least a portion of a syringe in a receptacle of a housing;
mechanically coupling a plunger of said syringe with a plunger slidable in a track of said housing such that movement of the plunger in a first direction causes movement of the syringe plunger in a second direction opposite to the first direction and movement of the syringe plunger in the first direction causes movement of the plunger in the second direction; and
urging one of the plunger and the syringe plunger in the first direction.

18. The method of claim 17, wherein said mechanical coupling comprises mating a thumb rest of said syringe plunger with a thumb rest of a second plunger slidable in a second track of said housing.

19. The method of claim 18, wherein said mating comprises sandwiching said thumb rest of said syringe plunger between two substantially parallel flanges of the thumb rest of said second plunger.

20. The method of claim 17, wherein said inserting comprises inserting a needle fitting of said syringe through an opening in said receptacle.
